(19) United States
(12) Patent Application Publication
(10) Pub. No.: US 2005/0118058 A1
(43) Pub. Date: Jun. 2, 2005

(54) Gentle preservative compositions for self-preserving solutions

(76) Inventors: Erning Xia, Penfield, NY (US); Joseph C. Salamone, Fairport, NY (US)

Correspondence Address: RITA D. VACCA;BAUSCH & LOMB INCORPORATED, ONE BAUSCH & LOMB PLACE, ROCHESTER, NY, 14604-2701, US

(21) Appl. No.: 10/725,049

(22) Filed: Dec. 1, 2003

(51) Int. Cl. A61L 12/14

(52) U.S. Cl. 422/28; 514/54; 514/840; 514/642

(57) ABSTRACT

Compositions are described which are useful in preserving any topically applied solution. Compositions including one or more cationic polysaccharides in solution in an amount effective to preserve contact lenses are also described, as well as methods of making and using such solutions.

### FIELD OF THE INVENTION

The present invention is directed toward the use of one or more cationic polysaccharides in the manufacture of gentle preservative systems. More particularly, the present invention is directed toward the use of compositions including one or more cationic polysaccharides to provide gentle preservation of ophthalmic solutions and medical devices.

### BACKGROUND OF THE INVENTION

Contact lenses in wide use today fall into two general categories, hard and soft. The hard or rigid corneal type lenses are formed from materials prepared by the polymerization of acrylic esters, such as poly(methyl methacrylate) (PMMA). The gel, hydrogel or soft type lenses are made by polymerizing such monomers as 2-hydroxyethyl methacrylate (HEMA) or, in the case of extended wear lenses, by polymerizing silicon-containing monomers or macromonomers. Both the hard and soft types of contact lenses are exposed to a broad spectrum of microbes during normal wear and become soiled relatively quickly. Contact lenses whether hard or soft therefore require routine cleaning and disinfecting. Failure to routinely clean and disinfect contact lenses properly can lead to a variety of problems ranging from mere discomfort when being worn to serious ocular infections. Ocular infections caused by virulent microbes such as Pseudomonas aeruginosa can lead to loss of the infected eye(s) if left untreated or if allowed to reach an advanced stage before initiating treatment.

U.S. Pat. No. 4,758,595 discloses a contact lens disinfectant and preservative containing a biguanide or a water-soluble salt thereof in combination with a buffer, preferably a borate buffer, e.g., boric acid, sodium borate, potassium tetraborate, potassium metaborate or mixtures of the same.

U.S. Pat. No. 4,361,548 discloses a contact lens disinfectant and preservative containing dilute aqueous solutions of a polymer; namely, dimethyldiallylammonium chloride (DMDAAC) having molecular weights ranging from about 10,000 to 1,000,000. Amounts of DMDAAC homopolymer as low as 0.00001 percent by weight may be employed when an enhancer, such as thimerosal, sorbic acid or phenylmercuric salt is used therewith. Although lens binding and concomitant eye tissue irritation with DMDAAC were reduced, it was found in some users to be above desirable clinical levels.

Despite the availability of various commercially available contact lens disinfecting systems such as heat, hydrogen peroxide, biguanides, polymeric biguanides, quaternary ammonium polyesters, amidoamines and other chemical agents, there continues to be a need for improved disinfecting and/or preserving systems. Such improved disinfecting and/or preserving systems include systems that are simple to use, are effective against a broad spectrum of microbes, are non-toxic and do not cause ocular irritation as the result of binding to the contact lens material. There is a particular need in the field of contact lens disinfection and ophthalmic composition preservation for safe and effective chemical agents with antimicrobial activity.

### SUMMARY OF THE INVENTION

The present invention relates to unique, gentle, self-preserving solutions such as for example but not limited to ophthalmic solutions and like solutions useful for topical application. Such self-preserving solutions may be useful for cleaning, soaking, rinsing, wetting and conditioning all types of contact lenses, including rigid permeable contact lenses, for nasal sprays, for ear drops, for eye drops and the like. It has been found that solutions containing compositions including one or more cationic polysaccharides exhibit excellent preservative effect, while also, in the case of contact lens solution use, increasing lens wearer comfort. The polysaccharide-containing compositions of the present invention are also useful for the preservation of ophthalmic solutions, pharmaceuticals, artificial tears, comfort drops and the like against microbial contamination.

The subject polysaccharide-containing compositions are effective preservatives useful in the manufacture of topical solutions that are non-toxic, simple to use and do not cause ocular irritation.

Accordingly, it is an object of the present invention to provide compositions with enhanced biocidal activity useful in the manufacture of self-preserving ophthalmic systems.

Another object of the present invention is to provide a method for using compositions with enhanced biocidal activity in the preservation of medical devices.

Another object of the present invention is to provide compositions with enhanced biocidal activity useful in ophthalmic systems to preserve contact lenses.

Another object of the present invention is to provide compositions with enhanced biocidal activity useful in preserving ophthalmic systems from microbial contamination.

Another object of the present invention is to provide compositions with enhanced biocidal activity useful in ophthalmic systems for preserving contact lenses with reduced or eliminated eye irritation.

Another object of the present invention is to provide a method of making gentle compositions having biocidal activity useful in preserving ophthalmic systems.

Still another object of the present invention is to provide a method of using gentle compositions with biocidal activity as preservative agents.

These and other objectives and advantages of the present invention, some of which are specifically described and others that are not, will become apparent from the detailed description and claims that follow.

### DETAILED DESCRIPTION OF THE INVENTION

The compositions of the present invention can be used with all contact lenses such as conventional hard and soft lenses, as well as rigid and soft gas permeable lenses. Such suitable lenses for use with compositions of the present invention include both hydrogel and non-hydrogel lenses, as well as silicone and fluorine-containing lenses. The term “soft contact lens” as used herein generally refers to those contact lenses that readily flex under small amounts of force. Typically, soft contact lenses are formulated from polymers having a certain proportion of repeat units derived from monomers such as 2-hydroxyethyl methacrylate and/or other hydrophilic monomers, typically crosslinked with a crosslinking agent. However, newer soft lenses, especially for extended wear, are being made from high-Dk silicone-containing materials.

Compositions of the present invention comprise one or more cationic polysaccharides. The polysaccharide-containing compositions of the present invention are useful in the production of self-preserving solutions. The self-preserving solutions are useful in preserving medical devices, pharmaceuticals, topically applied solutions and the like from microbial contamination. For example, the subject polysaccharide-containing compositions are useful in preserving contact lens care solutions employed in cleaning, soaking, rinsing and/or wetting contact lenses. Compositions of the present invention are preferably in solution in sufficient concentration to destroy harmful microorganisms and thus preserve the solution from microbial contamination throughout the intended shelf-life of the solution.

Compositions of the present invention in solution are physiologically compatible or “ophthalmically safe” for use with contact lenses. Ophthalmically safe as used herein means that a contact lens treated with or in the subject solution is generally suitable and safe for direct placement on the eye without rinsing. The subject solutions are safe and comfortable for daily contact with the eye via a contact lens that has been wetted with the solution. An ophthalmically safe solution has a tonicity and pH that is compatible with the eye and comprises materials, and amounts thereof, that are non-cytotoxic according to ISO (International Standards Organization) standards and U.S. FDA (Food and Drug Administration) regulations. Solutions of the present invention are sterile in that the absence of microbial contaminants in the product prior to release should be statistically demonstrated to the degree necessary for such products.

As noted above, compositions of the present invention include one or more cationic polysaccharides. One or more cationic polysaccharides are present in the subject compositions in a total amount of from approximately 0.001 to approximately 1.0 percent by weight based on the total weight of the composition, but more preferably from about 0.005 to about 0.1 percent by weight. Suitable cationic polysaccharides for use in compositions of the present invention include for example but are not limited to variations of polyquaternium-10 such as for example Polymer JR 125™ (Dow Chemical Company) having a 2 percent solution viscosity of 75-125 cPs and 1.5 to 2.2 percent nitrogen, Polymer JR 400™ (Dow Chemical Company) having a 2 percent solution viscosity of 300 to 500 cPs and 1.5 to 2.2 percent nitrogen, Polymer JR 30M™ (Dow Chemical Company) having a 1 percent solution viscosity of 1,000 to 2,500 cPs and 1.5 to 2.2 percent nitrogen, Polymer LR 400™ (Dow Chemical Company) having a 2 percent solution viscosity of 300 to 500 cPs and 0.8 to 1.1 percent nitrogen, Polymer LR 30M™ (Dow Chemical Company) having a 1 percent solution viscosity of 1,250 to 2,250 cPs and 0.8 to 1.1 percent nitrogen, and Polymer LK™ (Dow Chemical Company) having a 2 percent solution viscosity of 300 to 500 cPs and 0.8 to 1.1 percent nitrogen. The preferred cationic polysaccharide for use in the present invention is Polymer JR 125™ or Polymer JR 400™.

The above described cationic polysaccharides comprise a cellulosic backbone derived from natural, renewable resources. Cellulose is a straight chain polymer consisting of anhydroglucose sugars linked by β-1,4-bonds. Each anhydroglucose sugar monomer has three available hydroxyl (—OH) groups. Cellulose, in its original state, has a regular, hydrogen-bonded, crystalline structure, which is not readily water soluble. The addition of the hydroxyethyl groups on the cellulose backbone alters the polymer into a water soluble, easy to use product. Quaternization of hydroxyethylcellulose results in the creation of multiple cationic sites to which the anionic surface groups of microorganisms are attracted. Due to the multiple cationic sites of the cationic polysaccharides incorporated into compositions of the present invention, antimicrobial agents commonly used in ophthalmic solutions for preservation are not necessary. Accordingly, the use of ophthalmic solutions of the present invention cause less tissue irritation, less topical toxicity, provide greater user comfort and provide a broader biocidal spectrum.

In addition to one or more cationic polysaccharides, compositions of the present invention may optionally include one or more buffers, such as aminoalcohol buffers, such as for example but not limited to ethanolamine buffers. present in a total amount of from approximately 0.02 to approximately 3.0 percent by weight based on the total weight of the composition. Suitable aminoalcohol buffers include for example but are not limited to monoethanolamine (MEA), diethanolamine (DEA), triethanolamine (TEA), 2-amino-2-methyl-1,3-propanediol (AM PD), 2-dimethylamino-2-methyl-1-propanediol (DMAMP), 2-amino-2-ethylpropanol (AEP), 2-amino-1-butanol (AB) and 2-amino-2-methyl-1-propanol (AMP), but preferably MEA, DEA or TEA.

Compositions of the present invention may likewise optionally include one or more surfactants having known advantages in terms of cleaning efficacy and comfort. Surfactants may be present in the subject compositions in a total amount of from approximately 0.001 to approximately 5.0 percent by weight based on the total weight of the composition, but more preferably from about 0.1 to about 0.5 percent by weight. Suitable surfactants include for example but are not limited to polyethers based upon poly(ethylene oxide)-poly(propylene oxide)-poly(ethylene oxide), i.e., (PEO-PPO-PEO), or poly(propylene oxide)-poly(ethylene oxide)-poly(propylene oxide), i.e., (PPO-PEO-PPO), or a combination thereof. PEO-PPO-PEO and PPO-PEO-PPO are commercially available under the trade names PluronicsTM, R-Pluronics™, Tetronics™ and R-Tetronics™ (BASF Wyandotte Corp., Wyandotte, Mich.) and are further described in U.S. Pat. No. 4,820,352 incorporated herein in its entirety by reference. Suitable surfactants for use in the present composition should be soluble in the lens care solution, not become turbid, and should be non-irritating to eye tissues.

Optionally, it may be desirable to include one or more water-soluble viscosity agents in the subject compositions. Because of the demulcent effect of viscosity agents, the same have a tendency to enhance the lens wearer's comfort by means of a film on the lens surface cushioning impact against the eye. Suitable viscosity agents include for example but are not limited to cellulose polymers like hydroxyethyl or hydroxypropyl cellulose, carboxymethyl cellulose, povidone, polyvinyl alcohol and the like. Viscosity agents may be employed in amounts ranging from about 0.01 to about 4.0 weight percent or less.

Compositions of the present invention when in solution include one or more buffers, or a buffering system in addition to the aminoalcohol buffer, if any, to adjust the final pH of the solution. Suitable buffers include for example but are not limited to phosphate buffers, borate buffers, tris(hydroxymethyl)aminomethane (Tris) buffers, bis(2-hydroxyethyl)-imino-tris(hydroxymethyl)methane (bis-Tris) buffers, sodium bicarbonate, and combinations thereof. A suitable buffering system for example may include at least one phosphate buffer and at least one borate buffer, which buffering system has a buffering capacity of 0.01 to 0.5 mM, preferably 0.03 to 0.45, of 0.01 N of HCI and 0.01 to 0.3, preferably 0.025 to 0.25, of 0.01 N of NaOH to change the pH one unit. Buffering capacity is measured by a solution of the buffers only. The pH of lens care solutions of the present invention is preferably maintained within the range of 5.0 to 8.0, more preferably about 6.0 to 8.0, most preferably about 6.5 to 7.8.

Compositions of the present invention may likewise optionally include one or more tonicity agents to approximate the osmotic pressure of normal lachrymal fluids, which is equivalent to a 0.9 percent solution of sodium chloride or 2.5 percent glycerin solution. Examples of suitable tonicity agents include but are not limited to sodium and potassium chloride, dextrose, mannose, glycerin, calcium and magnesium chloride. These agents are typically used individually in amounts ranging from about 0.01 to 2.5 percent weight per volume and preferably, from about 0.2 to about 1.5 percent weight per volume. Preferably, the tonicity agent is employed in an amount to provide a final osmotic value of 200 to 450 mOsm/kg and more preferably between about 220 to about 350 mOsm/kg, and most preferably between about 220 to about 320 mOsm/kg.

Compositions of the present invention may also include one or more sequestering agents to bind metal ions, which in the case of ophthalmic solutions, might otherwise react with protein deposits and collect on contact lenses. Suitable sequestering agents include for example but are not limited to ethylenediaminetetraacetic acid (EDTA) and its salts. Sequestering agents are preferably used in amounts ranging from about 0.01 to about 0.2 weight percent. Surprisingly, it has been observed in formulations of the present invention that increased levels or amounts of EDTA and/or its salts in a formulation does not increase the formulation's preservative efficacy.

The compositions of the present invention are described in still greater detail in the examples that follow.

### EXAMPLE 1

### Preparation of Test Solutions

Sample solutions for testing were prepared in accordance with the formulations set forth below in Table 1.

TABLE 1
Test Solutions

| Ingredients | Solutions | | | |
|---|---|---|---|---|
| W/W Percent | 1 | 2 | 3 | 4 |
| Sodium Borate | 0.135 | 0.160 | 0.160 | 0.160 |
| Boric Acid | 1.000 | 1.400 | 1.400 | 1.400 |
| EDTA | 0.05 | 0.05 | 0.05 | 0.05 |
| Polymer JR 30M | 0.02 | 0.02 | 0.01 | 0.005 |
| pH | 7.0-7.4 | 7.11 | 7.11 | 7.11 |
| Osmolarity (mOsm/Kg) | 180-220 | 235 | 230 | 232 |
| Ingredients | Solutions | | | |
| W/W Percent | 5 | 6 | 7 | 8 |
| Sodium Borate | 0.160 | 0.160 | 0.160 | 0.160 |
| Boric Acid | 1.400 | 1.400 | 1.400 | 1.400 |
| EDTA | 0.10 | 0.05 | 0.025 | 0.05 |
| Polymer JR 30M | 0.02 | 0.02 | 0.02 | 0 |
| Polymer JR 400 | 0 | 0 | 0 | 0.05 |
| pH | 7.03 | 7.10 | 7.14 | 7.15 |
| Osmolarity (mOsm/Kg) | 242 | 235 | 233 | 235 |

| | |
|---|---|
| Ingredients | Solutions |

| W/W Percent | 9 | 10 |
|---|---|---|
| Sodium Borate | 0.160 | 0.160 |
| Boric Acid | 1.400 | 1.400 |
| EDTA | 0.05 | 0.05 |
| Polymer LR 400 | 0.05 | 0 |
| Polymer LK | 0 | 0.05 |
| pH | 7.15 | 7.15 |
| Osmolarity (mOsm/Kg) | 238 | 234 |

### EXAMPLE 2

### ISO/FDA Microbial Preservative Efficacy Testing of Test Solutions With Five of FDA/ISO Challenqe Microorganisms

Test solutions prepared in accordance with Example 1 above, were each tested for ISO/FDA microbial preservative efficacy using five FDA/ISO challenge microorganisms, i.e., three bacteria and two fungi. Acceptance criteria established for bacteria require that the number of viable bacteria, recovered per ml, is reduced by not less than 3.0 logs at 14 days. After the rechallenge at day 14, the concentration of bacteria shall be reduced by at least 3.0 logs by day 28. Acceptance criteria established for yeasts and molds require that the number of viable yeasts and molds, recovered per ml, remain at or below initial concentrations within an experimental error of ±0.5 logs within 14 days. After day 28, the concentration of mold and yeast shall remain at or below the concentrations after rechallenge within an experimental error of ±0.5 logs. Results of the ISO/FDA microbial preservative efficacy testing of the subject test solutions are set forth below in Table 2.

TABLE 2
Results of ISO/FDA Microbial Preservative Efficacy Testing

| | | Log Reduction of | | | | |
|---|---|---|---|---|---|---|
| Preservative Efficacy | | Solution | | | | |
| ISO Agent | Days | 1 | 2 | 3 | 4 | 5 |
| Staphylococcus aureus | 7 | 1.9 | 1.4 | 1.4 | 1.4 | 1.5 |
| (ATCC 6538) | 14 | >4.8 | 4.7 | >4.8 | >4.8 | >4.8 |
| | 21 | 1.6 | 1.1 | 1.1 | 1.1 | 1.1 |
| | 28 | >3.9 | >3.9 | >3.9 | >3.9 | 3.9 |
| Pseudomonas aeruginosa | 7 | >4.9 | >5.0 | >5.0 | 4.3 | >5.0 |
| (ATCC 9027) | 14 | >4.9 | >5.0 | >5.0 | >5.0 | >5.0 |
| | 21 | >3.9 | >3.8 | >3.8 | >3.8 | 3.8 |
| | 28 | >3.9 | >3.8 | >3.8 | >3.8 | 3.8 |
| Escherichia coli | 7 | >4.8 | 4.5 | 4.9 | 4.8 | 4.6 |
| (ATCC 8739) | 14 | >4.8 | >4.9 | >4.9 | >4.9 | >4.9 |
| | 21 | >3.9 | >3.8 | >3.8 | 3.8 | 3.2 |
| | 28 | >3.9 | 3.8 | 3.8 | >3.8 | >3.8 |
| Candida albicans | 7 | 2.2 | 2.3 | 1.8 | 1.9 | 1.7 |
| (ATCC 10231) | 14 | 4.2 | 4.3 | 3.1 | 3.1 | 2.8 |
| | 21 | 1.3 | 1.4 | 1.2 | 1.1 | 1.0 |
| | 28 | 2.5 | >3.9 | 3.9 | 3.9 | >3.9 |
| Aspergillus niger | 7 | 1.6 | 1.4 | 1.3 | 1.3 | 1.4 |
| (ATCC 16404) | 14 | 1.3 | 1.2 | 1.1 | 1.1 | 1.1 |
| | 21 | 0.6 | 0.4 | 0.4 | 0.4 | 0.3 |
| | 28 | 0.1 | 0.3 | 0.2 | 0.1 | 0.3 |
| | | Log Reduction of | | | | |
| Preservative Efficacy | | Solution | | | | |
| ISO Agent | Days | 6 | 7 | 8 | 9 | 10 |
| Staphylococcus aureus | 7 | 1.7 | 2.1 | 2.1 | >3.8 | 1.4 |
| (ATCC 6538) | 14 | >4.8 | >4.8 | >4.8 | 3.9 | 4.0 |
| | 21 | 1.4 | 1.4 | 2.3 | 1.4 | 1.6 |
| | 28 | >3.9 | >3.9 | >3.8 | >3.8 | >3.8 |
| Pseudomonas aeruginosa | 7 | >5.0 | >5.0 | >4.8 | >4.8 | >4.8 |
| (ATCC 9027) | 14 | >5.0 | >5.0 | >4.8 | 4.7 | >4.8 |
| | 21 | 3.8 | >3.8 | >3.8 | >3.8 | >3.8 |
| | 28 | 3.8 | >3.8 | >3.8 | >3.8 | >3.8 |
| Escherichia coli | 7 | >4.9 | 4.9 | 4.7 | 4.3 | 2.2 |
| (ATCC 8739) | 14 | >4.9 | >4.9 | >4.7 | 4.7 | 3.3 |
| | 21 | 3.8 | 3.8 | >3.8 | >3.8 | 2.2 |
| | 28 | >3.8 | >3.8 | >3.8 | 3.7 | 3.0 |
| Candida albicans | 7 | 2.1 | 2.6 | 2.1 | 0.5 | 0.3 |
| (ATCC 10231) | 14 | 3.7 | 4.9 | 4.8 | 1.8 | 1.8 |
| | 21 | 1.3 | 1.8 | 2.4 | 0.9 | 0.8 |
| | 28 | 3.9 | 3.9 | >3.9 | 2.7 | 2.6 |
| Aspergillus niger | 7 | 1.3 | 1.4 | 1.1 | 1.2 | 1.2 |
| (ATCC 16404) | 14 | 1.1 | 1.1 | 0.9 | 0.8 | 0.8 |
| | 21 | 0.4 | 0.4 | 0.3 | 0.2 | 0.3 |
| | 28 | 0.3 | 0.3 | −0.1 | 0.0 | 0.0 |

### EXAMPLE 3

### Preservative System Preparation

Sample solutions were prepared for testing in accordance with the preservative system formulations set forth below in Table 3.

TABLE 3
Test Solutions

| Ingredients | Solutions | | | | |
|---|---|---|---|---|---|
| W/W Percent | 11 | 12 | 13 | 14 | 15 |
| Sodium Borate | 0.160 | 0 | 0 | 0 | 0 |
| Boric Acid | 1.400 | 0 | 0 | 0 | 0 |
| EDTA | 0.05 | 0.05 | 0.05 | 0.05 | 0.50 |
| Polymer JR 30 M | 0.02 | 0.02 | 0 | 0 | 0 |
| Polymer JR 400 | 0 | 0 | 0.05 | 0 | 0 |
| Polymer LR 400 | 0 | 0 | 0 | 0.05 | 0 |
| Polymer LK | 0 | 0 | 0 | 0 | 0.05 |
| Triethanolamine | 0 | 0.934 | 0.934 | 0.934 | 0.934 |
| HCL (98%) | | | | | |
| Sodium Chloride | 0 | 0.340 | 0.340 | 0.340 | 0.340 |
| pH | 7.15 | 7.15 | 7.15 | 7.15 | 7.15 |
| Osmolarity (mOsm/Kg) | 237 | 212 | 241 | 203 | 209 |

### EXAMPLE 4

### ISO/FDA Microbial Preservative Efficacy Testinq of Test Solutions With Five of FDA/ISO Challenge Microorganisms

Test solutions prepared in accordance with Example 3 above, were each tested for ISO/FDA microbial preservative efficacy using five FDA/ISO challenge microorganisms, i.e., three bacteria and two fungi. Acceptance criteria established for bacteria require that the number of viable bacteria, recovered per ml, is reduced by not less than 3.0 logs at 14 days. After the rechallenge at day 14, the concentration of bacteria shall be reduced by at least 3.0 logs by day 28. Acceptance criteria established for yeasts and molds requires that the number of viable yeasts and molds, recovered per ml, remain at or below the initial concentrations within an experimental error of ±0.5 logs within 14 days. After day 28, the concentration of mold and yeast shall remain at or below the concentrations after rechallenge within an experimental error of ±0.5 logs. Results of the ISO/FDA microbial preservative efficacy testing of the subject test solutions are set forth below in Table 4.

TABLE 4
Results of ISO/FDA Microbial Preservative Efficacy Testing

| Preservative Efficacy | | | | | | |
|---|---|---|---|---|---|---|
| ISO Agent | Days | 11 | 12 | 13 | 14 | 15 |
| | | Log Reduction of | | | | |
| | | Solution | | | | |
| Staphylococcus aureus | 7 | 1.4 | >3.8 | >3.8 | >3.8 | 1.3 |
| (ATCC 6538) | 14 | >4.8 | 2.9 | 3.4 | 3.4 | 4.0 |
| | 21 | 1.8 | 0.7 | 1.3 | 1.1 | 1.3 |
| | 28 | >3.8 | 2.9 | 3.4 | 3.3 | >3.8 |
| Pseudomonas aeruginosa | 7 | >4.8 | ND | ND | ND | ND |
| (ATCC 9027) | 14 | >4.8 | 1.8 | 1.5 | 1.5 | 1.5 |
| | 21 | >3.8 | ND | ND | ND | ND |
| | 28 | >3.8 | ND | ND | ND | ND |
| Escherichia coli | 7 | >4.7 | 4.0 | 3.4 | >3.7 | >3.7 |
| (ATCC 8739) | 14 | >4.7 | 4.7 | >4.7 | 2.1 | 2.7 |
| | 21 | >3.8 | 3.5 | 3.8 | 2.1 | 0.9 |
| | 28 | >3.8 | >3.8 | >3.8 | 3.8 | ND |
| | | Log Reduction of | | | | |
| | | Sample | | | | |
| Candida albicans | 7 | 1.8 | 0.2 | 0.2 | 0.2 | 0.2 |
| (ATCC 10231) | 14 | 3.2 | 0.8 | 0.7 | 0.5 | 0.7 |
| | 21 | 1.6 | 0.5 | 0.5 | 0.5 | 0.5 |
| | 28 | 3.6 | 0.9 | 1.0 | 1.1 | 0.9 |
| Aspergillus niger | 7 | 1.2 | >2.6 | >2.6 | >2.6 | >2.6 |
| (ATCC 16404) | 14 | 0.9 | 0.1 | 0.0 | 0.0 | −0.1 |
| | 21 | 0.3 | −0.1 | 0.0 | −0.1 | 0.0 |
| | 28 | 0.1 | −0.1 | −0.1 | 0.0 | 0.0 |

Cationic polysaccharide containing compositions of the present invention are useful in contact lens care solutions for preserving contact lenses. A preserving amount of a cationic polysaccharide is an amount that will at least partially reduce the microorganism population in the formulations employed. Preferably, a preserving amount is that which will reduce the microbial burden of representative bacteria by two log orders in four hours and more preferably by one log order in one hour. Most preferably, a preserving amount is an amount that will eliminate the microbial burden on a contact lens when used according to its regimen for the recommended soaking time (FDA Chemical Disinfection Efficacy Test—July 1985 Contact Lens Solution Draft Guidelines). Unexpectedly, in the presence of one or more cationic polysaccharides, a antimicrobial agent is not required to achieve effective solution preservation.

As stated above, contact lenses are preserved by contacting the lenses with a solution containing an effective amount of one or more compositions of the present composition. Although this may be accomplished by simply soaking lenses in the subject solution, greater cleaning can be achieved if a few drops of the solution are initially placed on each side of the lens, and rubbing the lens for a period of time, for example, approximately 20 seconds. The lens can then be subsequently immersed within several milliliters of the subject solution. Preferably, the lens is permitted to soak in the solution for at least four hours. The lenses are then removed from the solution, rinsed with the same or a different solution, for example a cationic polysaccharide preserved isotonic saline solution made in accordance with the present invention, and then replaced on the eye.

Solutions containing one or more compositions of the present invention may be formulated into specific contact lens care products for use as customary in the field of ophthalmology. Such products include but are not limited to wetting solutions, soaking solutions, cleaning and conditioning solutions, as well as in-eye cleaning and conditioning solutions.

While the invention has been described in conjunction with specific examples thereof, this is illustrative only. Accordingly, many alternatives, modifications, and variations will be apparent to those skilled in the art in the light of the foregoing description and it is, therefore, intended to embrace all such alternatives, modifications, and variations as to fall within the spirit and scope of the appended claims.

1. Compositions comprising:
one or more cationic polysaccharides in solution in an amount effective for solution preservation.

2. Compositions comprising:
one or more cationic polysaccharides in an amount effective as a preserving agent.

3. The compositions of claim 1 or 2 wherein said one or more cationic polysaccharides are selected from the group consisting of variations of polyquaternium-10.

4. The compositions of claim 1 or 2 wherein said one or more cationic polysaccharides are selected from the group consisting of Polymer JR 125, Polymer JR 400, Polymer JR 30M, Polymer LR 400, Polymer LR 30 M and Polymer LK.

5. A method of producing compositions of claim 1 or 2 comprising:
combining one or more cationic polysaccharides in an amount effective for solution preservation.

6. The method of claim 5 wherein said one or more cationic polysaccharides are selected from the group consisting of variations of polyquaternium-10.

7. The method of claim 5 wherein said one or more cationic polysaccharides are selected from the group consisting of Polymer JR 125, Polymer JR 400, Polymer JR 30M, Polymer LR 400, Polymer LR 30 M and Polymer LK.

8. A solution comprising one or more compositions of claim 1 or 2.

9. The solution of claim 8 wherein said solution includes one or more buffers or buffering systems.

10. The solution of claim 8 wherein said solution includes one or more tonicity agents.

11. The solution of claim 8 wherein said solution includes one or more surfactants.

12. The solution of claim 8 wherein said solution includes one or more viscosity agents.

13. A method of using the solution of claim 8 comprising:
contacting a surface of a contact lens with said solution for a period of time suitable to eliminate a microbial burden on said contact lens.

14. A method of using the solution of claim 8 comprising:
contacting a surface of a medical device with said solution for a period of time suitable to eliminate a microbial burden on said medical device.

15. A method of producing the solution of claim 8 comprising:
adding an effective amount of one or more cationic polysaccharides to a solution.
